(19) United States
(12) Patent Application Publication
(10) Pub. No.: US 2025/0182449 A1
(43) Pub. Date: Jun. 5, 2025

(54) AUTOMATED VESSEL WALL SEGMENTATION SYSTEM AND METHOD

(71) Applicant: UNIVERSITY OF SOUTHERN CALIFORNIA, Los Angeles, CA (US)

(72) Inventors: Zhaoyang Fan, Los Angeles, CA (US); Dan Ruan, Los Angeles, CA (US); Jiayu Xiao, Los Angeles, CA (US); Hanyue Zhou, Los Angeles, CA (US)

(73) Assignee: UNIVERSITY OF SOUTHERN CALIFORNIA, Los Angeles, CA (US)

(21) Appl. No.: 18/843,996

(22) PCT Filed: Mar. 23, 2023
(86) PCT No.: PCT/US2023/016060

§ 371 Date: Sep. 4, 2024

Related U.S. Application Data
(60) Provisional application No. 63/323,004, filed on Mar. 23, 2022.

(51) Int. Cl. G06V 10/764; G06T 5/60; G06T 5/70; G06T 7/12; G06V 10/82

(52) U.S. Cl. CPC G06V 10/764; G06T 5/60; G06T 5/70; G06T 7/12; G06V 10/82; G06T 2207/10088; G06T 2207/20081; G06T 2207/20084; G06T 2207/20192; G06T 2207/30101; G06V 2201/031

(57) ABSTRACT

A system can comprise a processor and a non-transitory computer-readable storage devices storing computing instructions configured to run on the processor and cause the processor to perform receiving a magnetic resonance imaging (MRI) scan, feeding the MRI scan into a predictive algorithm, and outputting an improved MRI scan from the predictive algorithm.

### CROSS REFERENCE TO RELATED APPLICATIONS

This application claims priority to U.S. Provisional Appl. No. 63/323,004, entitled “AUTOMATED VESSEL WALL SEGMENTATION SYSTEM AND METHOD” and filed Mar. 23, 2022, which is herein incorporated by this reference in its entirety.

### STATEMENT AS TO FEDERALLY SPONSORED RESEARCH

This invention was made with government support under grant NIH R01 HL 147355 from the National Institutes of Health (NIH). The government has certain rights in this invention.

### TECHNICAL FIELD

This disclosure generally related to artificial intelligence and relates more specifically to neural networks used in magnetic resonance (MR) imaging.

### BACKGROUND

Stroke is a leading cause of morbidity and mortality in the US and worldwide and intracranial atherosclerosis disease (ICAD), which can be characterized by lipid deposition, inflammation, and remodeling in artery vessel walls, remains a major risk factor for stroke occurrence. Magnetic resonance (MR) vessel wall imaging (VWI) is an emerging non-invasive technology that can be useful in ICAD evaluation due to its high spatial resolution and dark-blood contrast. Quantitative assessment of atherosclerotic lesions based on MR-VWI may provide valuable insights into the severity of ICAD. For example, morphological measurements such as normalized wall index, arterial wall remodeling ratio, and plaque-to-wall contrast ratio have been shown to be useful imaging surrogates for plaque burden quantification in ICAD. These measurements often rely on accurate contouring of the vessel wall in a cross-sectional view for accuracy.

Generally speaking, vessel wall contouring is performed manually by a human specialist and can therefore be subject to high inter and intra observer variations and/or biases. These variations and/or biases can then induce high uncertainty on subsequent quantitative analysis on small intracranial arteries. Moreover, the presence of multiple ICAD lesions in a patient increases a time needed for specialist review, thereby exacerbating human errors due to fatigue.

Conventional automated or semi-automated vessel wall segmentation methods applied to MR-VWI images are generally based on explicit model fitting. For example, the shape of a whole carotid vessel can be approximated as elliptic, and can then be translated, deformed, and rotated iteratively to fit an outer vessel wall boundary. Model fitting, though, can have many disadvantages. For example, model fitting algorithms for vessel wall segmentation can suffer from long computation times (e.g., when iterative model fitting is used) and model misfits can occur when shape assumptions are violated.

Recent efforts have created deep neural networks that perform automated vessel wall segmentation. While these neural network approaches can be effective for large vessels, they often struggle with smaller lumens (e.g., intracranial vessels) due, at least in part, to the signal and contrast strength of smaller lumens being low or disrupted.

Therefore, there is a need for an automated system and method to improve accuracy, consistency, and efficiency of segmentation in MR images.

### BRIEF DESCRIPTION OF THE DRAWINGS

To facilitate further description of the embodiments, the following drawings are provided in which:

FIG. 1 illustrates a front elevational view of a computer system that is suitable for implementing various embodiments of the systems disclosed in FIGS. 3 and 5;

FIG. 2 illustrates a representative block diagram of an example of the elements included in the circuit boards inside a chassis of the computer system of FIG. 1;

FIG. 3 illustrates a representative block diagram of a system, according to an embodiment;

FIG. 4 illustrates a flowchart for a method, according to an embodiment; and

FIG. 5 illustrates a representative block diagram of a system, according to an embodiment.

For simplicity and clarity of illustration, the drawing figures illustrate the general manner of construction, and descriptions and details of well-known features and techniques may be omitted to avoid unnecessarily obscuring the present disclosure. Additionally, elements in the drawing figures are not necessarily drawn to scale. For example, the dimensions of some of the elements in the figures may be exaggerated relative to other elements to help improve understanding of embodiments of the present disclosure. The same reference numerals in different figures denotes the same elements.

The terms “first,” “second,” “third,” “fourth,” and the like in the description and in the claims, if any, are used for distinguishing between similar elements and not necessarily for describing a particular sequential or chronological order. It is to be understood that the terms so used are interchangeable under appropriate circumstances such that the embodiments described herein are, for example, capable of operation in sequences other than those illustrated or otherwise described herein. Furthermore, the terms “include,” and “have,” and any variations thereof, are intended to cover a non-exclusive inclusion, such that a process, method, system, article, device, or apparatus that comprises a list of elements is not necessarily limited to those elements but may include other elements not expressly listed or inherent to such process, method, system, article, device, or apparatus.

The terms “left,” “right,” “front,” “back,” “top,” “bottom,” “over,” “under,” and the like in the description and in the claims, if any, are used for descriptive purposes and not necessarily for describing permanent relative positions. It is to be understood that the terms so used are interchangeable under appropriate circumstances such that the embodiments of the apparatus, methods, and/or articles of manufacture described herein are, for example, capable of operation in other orientations than those illustrated or otherwise described herein.

The terms “couple,” “coupled,” “couples,” “coupling,” and the like should be broadly understood and refer to connecting two or more elements mechanically and/or otherwise. Two or more electrical elements may be electrically coupled together, but not be mechanically or otherwise coupled together. Coupling may be for any length of time, e.g., permanent or semi-permanent or only for an instant. “Electrical coupling” and the like should be broadly understood and include electrical coupling of all types. The absence of the word “removably,” “removable,” and the like near the word “coupled,” and the like does not mean that the coupling, etc. in question is or is not removable.

As defined herein, two or more elements are “integral” if they are comprised of the same piece of material. As defined herein, two or more elements are “non-integral” if each is comprised of a different piece of material.

As defined herein, “real-time” can, in some embodiments, be defined with respect to operations carried out as soon as practically possible upon occurrence of a triggering event. A triggering event can include receipt of data necessary to execute a task or to otherwise process information. Because of delays inherent in transmission and/or in computing speeds, the term “real time” encompasses operations that occur in “near” real time or somewhat delayed from a triggering event. In a number of embodiments, “real time” can mean real time less a time delay for processing (e.g., determining) and/or transmitting data. The particular time delay can vary depending on the type and/or amount of the data, the processing speeds of the hardware, the transmission capability of the communication hardware, the transmission distance, etc. However, in some embodiments, the time delay can be less than approximately one second, two seconds, five seconds, or ten seconds.

As defined herein, “approximately” can, in some embodiments, mean within plus or minus ten percent of the stated value. In other embodiments, “approximately” can mean within plus or minus five percent of the stated value. In further embodiments, “approximately” can mean within plus or minus three percent of the stated value. In yet other embodiments, “approximately” can mean within plus or minus one percent of the stated value.

### DETAILED DESCRIPTION

Some embodiments can include a system. The system can comprise a processor; and a non-transitory computer-readable storage devices storing computing instructions configured to run on the processor and cause the processor to perform receiving a magnetic resonance imaging (MRI) scan; feeding the MRI scan into a predictive algorithm; and outputting an improved MRI scan from the predictive algorithm.

Some embodiments can include a method. The method can comprise receiving a magnetic resonance imaging (MRI) scan; feeding the MRI scan into a predictive algorithm; and outputting an improved MRI scan from the predictive algorithm.

Various embodiments can include a method. The method can comprise receiving data representative of an image of at least one of (i) a lumen, (ii) a background, and (iii) a vessel wall at a first input of a first convolution layer, the first convolution layer having a first input and a first output connected to a batch normalization layer and providing a first output data at the first output; normalizing, by the batch normalization layer, the first output data to create normalized output data; providing, by the batch normalization layer, the normalized output data to a second convolution layer having a second input to receive the normalized output data and having a second output; maximizing, by a skip connection including a neural network connected between the first input and the second input, a function comprising at least a fidelity term, a first regularization term, and a second regularization term and providing the maximized function to the second convolution layer; calculating, by the second convolution layer, a second output data based on the normalized output data and the function, the second output data representing a predicted classification of the pixel as corresponding to at least one of (i) the lumen, (ii) the background, and (iii) the vessel wall; and providing, by the second convolution layer, the second output data at the second output.

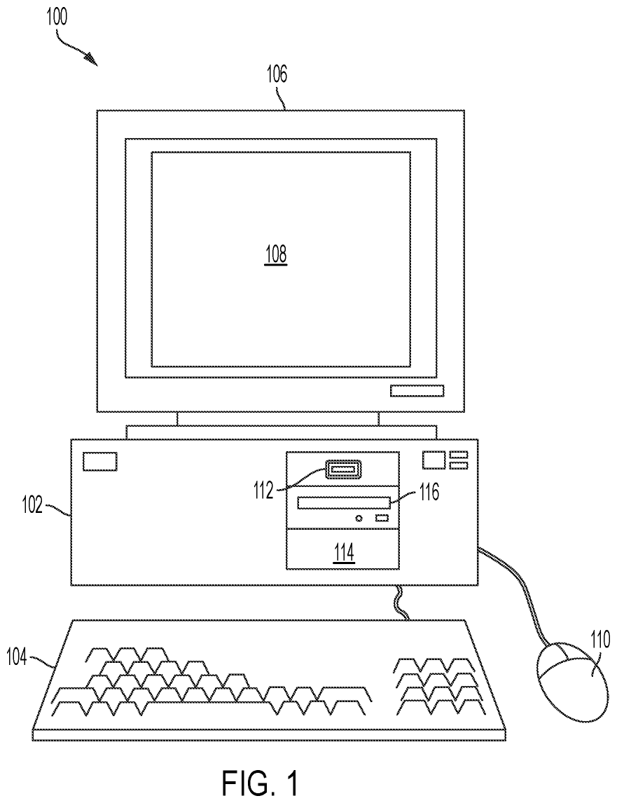

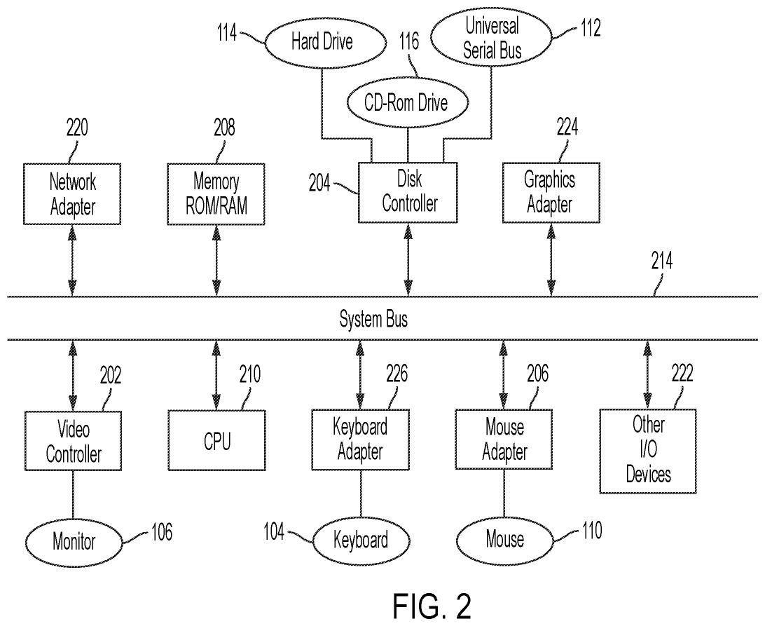

Turning to the drawings, FIG. 1 illustrates an exemplary embodiment of a computer system 100, all of which or a portion of which can be suitable for (i) implementing part or all of one or more embodiments of the techniques, methods, and systems and/or (ii) implementing and/or operating part or all of one or more embodiments of the memory storage modules described herein. As an example, a different or separate one of a chassis 102 (and its internal components) can be suitable for implementing part or all of one or more embodiments of the techniques, methods, and/or systems described herein. Furthermore, one or more elements of computer system 100 (e.g., a monitor 106, a keyboard 104, and/or a mouse 110, etc.) also can be appropriate for implementing part or all of one or more embodiments of the techniques, methods, and/or systems described herein. Computer system 100 can comprise chassis 102 containing one or more circuit boards (not shown), a Universal Serial Bus (USB) port 112, a Compact Disc Read-Only Memory (CD-ROM) and/or Digital Video Disc (DVD) drive 116, and a hard drive 114. A representative block diagram of the elements included on the circuit boards inside chassis 102 is shown in FIG. 2. A central processing unit (CPU) 210 in FIG. 2 is coupled to a system bus 214 in FIG. 2. In various embodiments, the architecture of CPU 210 can be compliant with any of a variety of commercially distributed architecture families.

Continuing with FIG. 2, system bus 214 also is coupled to a memory storage unit 208, where memory storage unit 208 can comprise (i) non-volatile memory, such as, for example, read only memory (ROM) and/or (ii) volatile memory, such as, for example, random access memory (RAM). The non-volatile memory can be removable and/or non-removable non-volatile memory. Meanwhile, RAM can include dynamic RAM (DRAM), static RAM (SRAM), etc. Further, ROM can include mask-programmed ROM, programmable ROM (PROM), one-time programmable ROM (OTP), erasable programmable read-only memory (EPROM), electrically erasable programmable ROM (EEPROM) (e.g., electrically alterable ROM (EAROM) and/or flash memory), etc. In these or other embodiments, memory storage unit 208 can comprise (i) non-transitory memory and/or (ii) transitory memory.

In some embodiments, all or a portion of memory storage unit 208 can be referred to as memory storage module(s) and/or memory storage device(s). In various examples, portions of the memory storage module(s) of the various embodiments disclosed herein (e.g., portions of the non-volatile memory storage module(s)) can be encoded with a boot code sequence suitable for restoring computer system 100 (FIG. 1) to a functional state after a system reset. In addition, portions of the memory storage module(s) of the various embodiments disclosed herein (e.g., portions of the non-volatile memory storage module(s)) can comprise microcode such as a Basic Input-Output System (BIOS) operable with computer system 100 (FIG. 1). In the same or different examples, portions of the memory storage module(s) of the various embodiments disclosed herein (e.g., portions of the non-volatile memory storage module(s)) can comprise an operating system, which can be a software program that manages the hardware and software resources of a computer and/or a computer network. The BIOS can initialize and test components of computer system 100 (FIG. 1) and load the operating system. Meanwhile, the operating system can perform basic tasks such as, for example, controlling and allocating memory, prioritizing the processing of instructions, controlling input and output devices, facilitating networking, and managing files. Exemplary operating systems can comprise one of the following: (i) Microsoft® Windows® operating system (OS) by Microsoft Corp. of Redmond, Washington, United States of America, (ii) Mac® OS X by Apple Inc. of Cupertino, California, United States of America, (iii) UNIX® OS, and (iv) Linux® OS. Further exemplary operating systems can comprise one of the following: (i) the iOS® operating system by Apple Inc. of Cupertino, California, United States of America, (ii) the Blackberry® operating system by Research In Motion (RIM) of Waterloo, Ontario, Canada, (iii) the WebOS operating system by LG Electronics of Seoul, South Korea, (iv) the Android™ operating system developed by Google, of Mountain View. California, United States of America, (v) the Windows Mobile™ operating system by Microsoft Corp. of Redmond, Washington, United States of America, or (vi) the Symbian™ operating system by Accenture PLC of Dublin, Ireland.

As used herein, “processor” and/or “processing module” means any type of computational circuit, such as but not limited to a microprocessor, a microcontroller, a controller, a complex instruction set computing (CISC) microprocessor, a reduced instruction set computing (RISC) microprocessor, a very long instruction word (VLIW) microprocessor, a graphics processor, a digital signal processor, or any other type of processor or processing circuit capable of performing the desired functions. In some examples, the one or more processing modules of the various embodiments disclosed herein can comprise CPU 210.

Alternatively, or in addition to, the systems and procedures described herein can be implemented in hardware, or a combination of hardware, software, and/or firmware. For example, one or more application specific integrated circuits (ASICs) can be programmed to carry out one or more of the systems and procedures described herein. For example, one or more of the programs and/or executable program components described herein can be implemented in one or more ASICs. In some embodiments, an application specific integrated circuit (ASIC) can comprise one or more processors or microprocessors and/or memory blocks or memory storage.

In the depicted embodiment of FIG. 2, various I/O devices such as a disk controller 204, a graphics adapter 224, a video controller 202, a keyboard adapter 226, a mouse adapter 206, a network adapter 220, and other I/O devices 222 can be coupled to system bus 214. Keyboard adapter 226 and mouse adapter 206 are coupled to keyboard 104 (FIGS. 1-2) and mouse 110 (FIGS. 1-2), respectively, of computer system 100 (FIG. 1). While graphics adapter 224 and video controller 202 are indicated as distinct units in FIG. 2, video controller 202 can be integrated into graphics adapter 224, or vice versa in other embodiments. Video controller 202 is suitable for monitor 106 (FIGS. 1-2) to display images on a screen 108 (FIG. 1) of computer system 100 (FIG. 1). Disk controller 204 can control hard drive 114 (FIGS. 1-2), USB port 112 (FIGS. 1-2), and CD-ROM drive 116 (FIGS. 1-2). In other embodiments, distinct units can be used to control each of these devices separately.

Network adapter 220 can be suitable to connect computer system 100 (FIG. 1) to a computer network by wired communication (e.g., a wired network adapter) and/or wireless communication (e.g., a wireless network adapter). In some embodiments, network adapter 220 can be plugged or coupled to an expansion port (not shown) in computer system 100 (FIG. 1). In other embodiments, network adapter 220 can be built into computer system 100 (FIG. 1). For example, network adapter 220 can be built into computer system 100 (FIG. 1) by being integrated into the motherboard chipset (not shown) or implemented via one or more dedicated communication chips (not shown), connected through a PCI (peripheral component interconnector) or a PCI express bus of computer system 100 (FIG. 1) or USB port 112 (FIG. 1).

Returning now to FIG. 1, although many other components of computer system 100 are not shown, such components and their interconnection are well known to those of ordinary skill in the art. Accordingly, further details concerning the construction and composition of computer system 100 and the circuit boards inside chassis 102 are not discussed herein.

Meanwhile, when computer system 100 is running, program instructions (e.g., computer instructions) stored on one or more of the memory storage module(s) of the various embodiments disclosed herein can be executed by CPU 210 (FIG. 2). At least a portion of the program instructions, stored on these devices, can be suitable for carrying out at least part of the techniques and methods described herein.

Further, although computer system 100 is illustrated as a desktop computer in FIG. 1, there can be examples where computer system 100 may take a different form factor while still having functional elements similar to those described for computer system 100. In some embodiments, computer system 100 may comprise a single computer, a single server, or a cluster or collection of computers or servers, or a cloud of computers or servers. Typically, a cluster or collection of servers can be used when the demand on computer system 100 exceeds the reasonable capability of a single server or computer. In certain embodiments, computer system 100 may comprise a portable computer, such as a laptop computer. In certain other embodiments, computer system 100 may comprise a mobile electronic device, such as a smartphone. In certain additional embodiments, computer system 100 may comprise an embedded system.

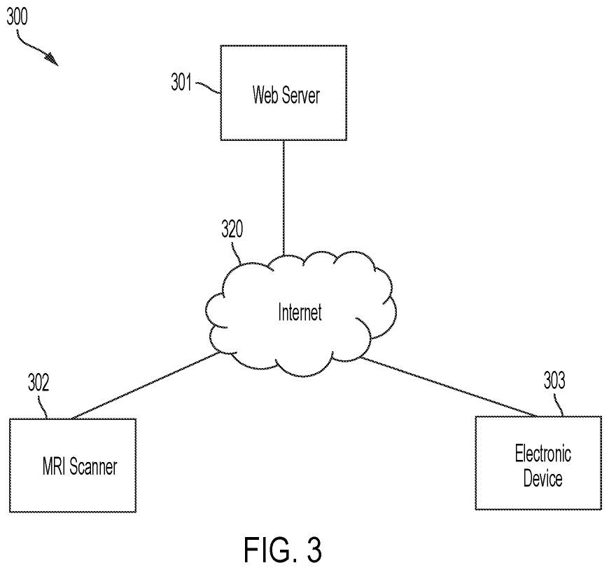

Turning ahead in the drawings, FIG. 3 illustrates a block diagram of a system 300 that can be employed for automated vessel wall segmentation, as described in greater detail below. System 300 is merely exemplary and embodiments of the system are not limited to the embodiments presented herein. System 300 can be employed in many different embodiments or examples not specifically depicted or described herein. In some embodiments, certain elements or modules of system 300 can perform various procedures, processes, and/or activities. In these or other embodiments, the procedures, processes, and/or activities can be performed by other suitable elements or modules of system 300.

Generally, therefore, system 300 can be implemented with hardware and/or software, as described herein. In some embodiments, part or all of the hardware and/or software can be conventional, while in these or other embodiments, part or all of the hardware and/or software can be customized (e.g., optimized) for implementing part or all of the functionality of system 300 described herein.

In some embodiments, system 300 can include a web server 301, an MRI scanner 302, and/or an electronic device 303. Web server 301, MRI scanner 302, and/or electronic device 303 can each be and/or incorporate a computer system, such as computer system 100 (FIG. 1), as described above, and can each be a single computer, a single server, or a cluster or collection of computers or servers, or a cloud of computers or servers. In another embodiment, a single computer system can host each of two or more of web server 301, MRI scanner 302, and/or electronic device 303. Additional details regarding web server 301, MRI scanner 302, and/or electronic device 303 are described herein.

Generally speaking, MRI scanner 302 can comprise a machine configured to create a magnetic field and use radio waves to produce internal images of the body (e.g., blood vessels or other biological lumens). In various embodiments, MRI scanner 302 can comprise a magnet configured to cast a magnetic field onto a patient. In some embodiments, a magnetic field can align hydrogen atoms in a patient's body. In some embodiments, MRI scanner 302 can comprise a radio frequency (RF) coil. In further embodiments, a RF coil can be configured to generate RF waves that excite hydrogen atoms in a patient's body. In some embodiments, a RF coil can be configured to receive RF signals emitted from hydrogen atoms after excitation. Received RF signals can then be processed into images displayed on web server 301, MRI scanner 302, and/or electronic device 303 using various mathematical techniques (e.g., by using a Fourier transform). In some embodiments, MRI scanner 302 can comprise shielding (e.g., magnetic shielding) configured to prevent outside signals from interfering with an MRI scan of a patient.

In some embodiments, web server 301, MRI scanner 302, and/or electronic device 303 can be mobile devices. A mobile electronic device can refer to a portable electronic device (e.g., an electronic device easily conveyable by hand by a person of average size) with the capability to present audio and/or visual data (e.g., text, images, videos, music, etc.). For example, a mobile electronic device can comprise at least one of a digital media player, a cellular telephone (e.g., a smartphone), a personal digital assistant, a handheld digital computer device (e.g., a tablet personal computer device), a laptop computer device (e.g., a notebook computer device, a netbook computer device), a wearable user computer device, or another portable computer device with the capability to present audio and/or visual data (e.g., images, videos, music, etc.). Thus, in many examples, a mobile electronic device can comprise a volume and/or weight sufficiently small as to permit the mobile electronic device to be easily conveyable by hand. For example, in some embodiments, a mobile electronic device can occupy a volume of less than or equal to approximately 1790 cubic centimeters, 2434 cubic centimeters, 2876 cubic centimeters, 4056 cubic centimeters, and/or 5752 cubic centimeters. Further, in these embodiments, a mobile electronic device can weigh less than or equal to 15.6 Newtons, 17.8 Newtons, 22.3 Newtons, 31.2 Newtons, and/or 44.5 Newtons.

Exemplary mobile electronic devices can comprise (i) an iPod®, iPhone®, iTouch®, iPad®, MacBook@) or similar product by Apple Inc. of Cupertino, California, United States of America, (ii) a Blackberry® R or similar product by Research in Motion (RIM) of Waterloo, Ontario, Canada, (iii) a Lumia® or similar product by the Nokia Corporation of Keilaniemi, Espoo, Finland, and/or (iv) a Galaxy™ or similar product by the Samsung Group of Samsung Town, Seoul, South Korea. Further, in the same or different embodiments, a mobile electronic device can comprise an electronic device configured to implement one or more of (i) the iPhone® operating system by Apple Inc. of Cupertino, California, United States of America, (ii) the Blackberry® operating system by Research In Motion (RIM) of Waterloo, Ontario, Canada, (iii) the Palm® operating system by Palm, Inc. of Sunnyvale, California, United States, (iv) the Android™ operating system developed by the Open Handset Alliance, (v) the Windows Mobile™ operating system by Microsoft Corp. of Redmond, Washington, United States of America, or (vi) the Symbian™ operating system by Nokia Corp. of Keilaniemi, Espoo, Finland.

Further still, the term “wearable user computer device” as used herein can refer to an electronic device with the capability to present audio and/or visual data (e.g., text, images, videos, music, etc.) that is configured to be worn by a user and/or mountable (e.g., fixed) on the user of the wearable user computer device (e.g., sometimes under or over clothing; and/or sometimes integrated with and/or as clothing and/or another accessory, such as, for example, a hat, eyeglasses, a wrist watch, shoes, etc.). In many examples, a wearable user computer device can comprise a mobile electronic device, and vice versa. However, a wearable user computer device does not necessarily comprise a mobile electronic device, and vice versa.

In specific examples, a wearable user computer device can comprise a head mountable wearable user computer device (e.g., one or more head mountable displays, one or more eyeglasses, one or more contact lenses, one or more retinal displays, etc.) or a limb mountable wearable user computer device (e.g., a smart watch). In these examples, a head mountable wearable user computer device can be mountable in close proximity to one or both eyes of a user of the head mountable wearable user computer device and/or vectored in alignment with a field of view of the user.

In more specific examples, a head mountable wearable user computer device can comprise (i) Google Glass™ product or a similar product by Google Inc. of Menlo Park, California, United States of America; (ii) the Eye Tap™ product, the Laser Eye Tap™ product, or a similar product by ePI Lab of Toronto, Ontario, Canada, and/or (iii) the Raptyr™ product, the STAR 1200™ product, the Vuzix Smart Glasses M100™ product, or a similar product by Vuzix Corporation of Rochester, New York, United States of America. In other specific examples, a head mountable wearable user computer device can comprise the Virtual Retinal Display™ product, or similar product by the University of Washington of Seattle, Washington, United States of America. Meanwhile, in further specific examples, a limb mountable wearable user computer device can comprise the iWatch™ product, or similar product by Apple Inc. of Cupertino, California, United States of America, the Galaxy Gear or similar product of Samsung Group of Samsung Town, Seoul, South Korea, the Moto 360 product or similar product of Motorola of Schaumburg, Illinois, United States of America, and/or the Zip™ product, One™ product, Flex™ product, Charge™ product, Surge™ product, or similar product by Fitbit Inc. of San Francisco, California, United States of America.

A mobile MRI scanner can refer to an MRI scanner that can be moved and/or is mounted on wheels. In various embodiments, a mobile MRI scanner can be mounted on a trailer and/or towed by an automobile. In these or other embodiments, an MRI scanner can be brought to a patient's bedside on a cart. Exemplary mobile MRI scanners include the Hyperfine Swoop™ and/or various non-mobile MRI scanners mounted on wheels.

In some embodiments, system 300 can comprise a graphical user interface (“GUI”). In the same or different embodiments, all or a part of a GUI can be part of and/or displayed by one or more of web server 301, MRI scanner 302, and/or electronic device 303. In some embodiments, a GUI can comprise text and/or graphics (image) based user interfaces. In the same or different embodiments, a GUI can comprise a heads up display (“HUD”). When a GUI comprises a HUD, a GUI can be projected onto a medium (e.g., glass, plastic, etc.), displayed in midair as a hologram, or displayed on a display (e.g., monitor 106 (FIG. 1)). In various embodiments, a GUI can be color, black and white, and/or greyscale. In some embodiments, a GUI can comprise a software application running on a computer system, such as computer system 100 (FIG. 1), web server 301, MRI scanner 302, and/or electronic device 303. In the same or different embodiments, a GUI can comprise a website accessed by electronic device 303 through internet 320. In some embodiments, a GUI can comprise a rotatable and/or navigable display of a vessel or some other interior region and/or organ in a patient's body. In the same or different embodiments, a GUI can be displayed as or on a virtual reality (VR) and/or augmented reality (AR) system or display (e.g., a head mounted electronic device and/or an AR scene displayed on a computer system). In some embodiments, an interaction with a GUI can comprise a click, a look, a selection, a grab, a view, a purchase, a bid, a swipe, a pinch, a reverse pinch, etc.

In some embodiments, web server 301, MRI scanner 302, and/or electronic device 303 can each comprise one or more input devices (e.g., one or more keyboards, one or more keypads, one or more pointing devices such as a computer mouse or computer mice, one or more touchscreen displays, a microphone, etc.), and/or can each comprise one or more display devices (e.g., one or more monitors, one or more touch screen displays, projectors, etc.). In these or other embodiments, one or more of the input device(s) can be similar or identical to keyboard 104 (FIG. 1) and/or a mouse 110 (FIG. 1). Further, one or more of the display device(s) can be similar or identical to monitor 106 (FIG. 1) and/or screen 108 (FIG. 1). The input device(s) and the display device(s) can be coupled to the processing module(s) and/or the memory storage module(s) of web server 301, MRI scanner 302, and/or electronic device 303 in a wired manner and/or a wireless manner, and the coupling can be direct and/or indirect, as well as locally and/or remotely. As an example of an indirect manner (which may or may not also be a remote manner), a keyboard-video-mouse (KVM) switch can be used to couple the input device(s) and the display device(s) to the processing module(s) and/or the memory storage module(s). In some embodiments, the KVM switch also can be part of web server 301, MRI scanner 302, and/or electronic device 303. In a similar manner, the processing module(s) and the memory storage module(s) can be local and/or remote to each other.

In some embodiments, web server 301, MRI scanner 302, and/or electronic device 303 can be configured to communicate with each other. In some embodiments, web server 301, MRI scanner 302, and/or electronic device 303 can communicate or interface (e.g., interact) with each other through a network (e.g., through internet 330). In some embodiments, Internet 330 can be an intranet that is not open to the public. In further embodiments, internet 330 can be a mesh network of individual systems. In various embodiments, one or more of web server 301, MRI scanner 302, and/or electronic device 303 can use one or more standard communication protocols to communicate through internet 320. In further embodiments, web server 301 and/or MRI scanner 302 (and/or the software used by such systems) can refer to a back end of system 300 operated by an operator and/or administrator of system 300 and electronic device 303 (and/or the software used by such systems) can refer to a front end of system 300 used by a patient and/or physician. In these or other embodiments, the operator and/or administrator of system 300 can manage system 300, the processing module(s) of system 300, and/or the memory storage module(s) of system 300 using the input device(s) and/or display device(s) of system 300.

Meanwhile, in some embodiments, web server 301, MRI scanner 302, and/or electronic device 303 also can be configured to communicate with one or more databases. The one or more databases can comprise an MRI scan database that contains information about various types of MRI scans. For example, data describing a k-space for past or current MRI scans can be saved in the one or more databases. In some embodiments, data can be deleted from a database when it becomes older than a maximum age. In some embodiments, a maximum age can be determined by an administrator of system 300. In various embodiments, data collected by MRI scanner 302 in real-time can be streamed to a database for storage.

In some embodiments, one or more databases can be stored on one or more memory storage modules (e.g., non-transitory memory storage module(s)), which can be similar or identical to the one or more memory storage module(s) (e.g., non-transitory memory storage module(s)) described above with respect to computer system 100 (FIG. 1). Also, in some embodiments, for any particular database of the one or more databases, that particular database can be stored on a single memory storage module of the memory storage module(s), and/or the non-transitory memory storage module(s) storing the one or more databases or the contents of that particular database can be spread across multiple ones of the memory storage module(s) and/or non-transitory memory storage module(s) storing the one or more databases, depending on the size of the particular database and/or the storage capacity of the memory storage module(s) and/or non-transitory memory storage module(s). In various embodiments, databases can be stored in a cache (e.g., MegaCache) for immediate retrieval on-demand. The one or more databases can each comprise a structured (e.g., indexed) collection of data and can be managed by any suitable database management systems configured to define, create, query, organize, update, and manage database(s). Exemplary database management systems can include MySQL (Structured Query Language) Database, PostgreSQL Database, Microsoft SQL Server Database, Oracle Database, SAP (Systems, Applications, & Products) Database, IBM DB2 Database, and/or NoSQL Database.

Meanwhile, communication between web server 301, MRI scanner 302, electronic device 303, and/or the one or more databases can be implemented using any suitable manner of wired and/or wireless communication. Accordingly, system 300 can comprise any software and/or hardware components configured to implement the wired and/or wireless communication. Further, the wired and/or wireless communication can be implemented using any one or any combination of wired and/or wireless communication network topologies (e.g., ring, line, tree, bus, mesh, star, daisy chain, hybrid, etc.) and/or protocols (e.g., personal area network (PAN) protocol(s), local area network (LAN) protocol(s), wide area network (WAN) protocol(s), cellular network protocol(s), powerline network protocol(s), etc.). Exemplary PAN protocol(s) can comprise Bluetooth, Zigbee, Wireless Universal Serial Bus (USB), Z-Wave, etc.; exemplary LAN and/or WAN protocol(s) can comprise Institute of Electrical and Electronic Engineers (IEEE) 802.3 (also known as Ethernet), IEEE 802.11 (also known as WiFi), etc.; and exemplary wireless cellular network protocol(s) can comprise Global System for Mobile Communications (GSM), General Packet Radio Service (GPRS), Code Division Multiple Access (CDMA), Evolution-Data Optimized (EV-DO), Enhanced Data Rates for GSM Evolution (EDGE), Universal Mobile Telecommunications System (UMTS), Digital Enhanced Cordless Telecommunications (DECT), Digital AMPS (IS-136/Time Division Multiple Access (TDMA)), Integrated Digital Enhanced Network (iDEN), Evolved High-Speed Packet Access (HSPA+), Long-Term Evolution (LTE), WiMAX, etc. The specific communication software and/or hardware implemented can depend on the network topologies and/or protocols implemented, and vice versa. In various embodiments, exemplary communication hardware can comprise wired communication hardware including, for example, one or more data buses, such as, for example, universal serial bus(es), one or more networking cables, such as, for example, coaxial cable(s), optical fiber cable(s), and/or twisted pair cable(s), any other suitable data cable, etc. Further exemplary communication hardware can comprise wireless communication hardware including, for example, one or more radio transceivers, one or more infrared transceivers, etc. Additional exemplary communication hardware can comprise one or more networking components (e.g., modulator-demodulator components, gateway components, etc.).

The techniques described herein can provide a practical application and several technological improvements. In some embodiments, the techniques described herein can provide for a more quickly generated, more accurate, and/or more precise image of an MRI scan. The techniques described herein can provide a significant improvement over conventional approaches of producing an MRI scan (e.g., manual interpretation by a specialist). In some embodiments, the techniques described herein can beneficially make determinations based on dynamic information that includes past MRI scans and/or MRI scans that have occurred during the same day as a patient's scan. In this way, the techniques described herein can avoid problems with stale and/or outdated predictive algorithms by continually updating. In a number of embodiments, the techniques described herein can advantageously provide an improved MRI scan by minimizing and/or removing artifacts that are created by the MRI scan and not present in vivo.

In some embodiments, the techniques described herein can be used in a way that cannot be reasonably performed using manual techniques or the human mind. For example, the human mind cannot create MRI images because it does not emit a magnetic field or radio waves strong enough to produce an MRI scan. Further, implementing and/or training a predictive algorithm can use extensive data inputs analyzed at speeds that cannot reasonably be performed in the mind of a human.

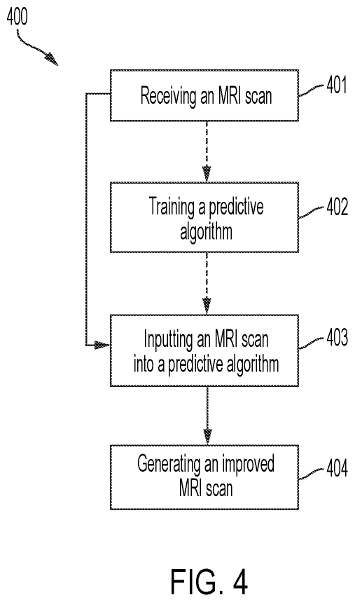

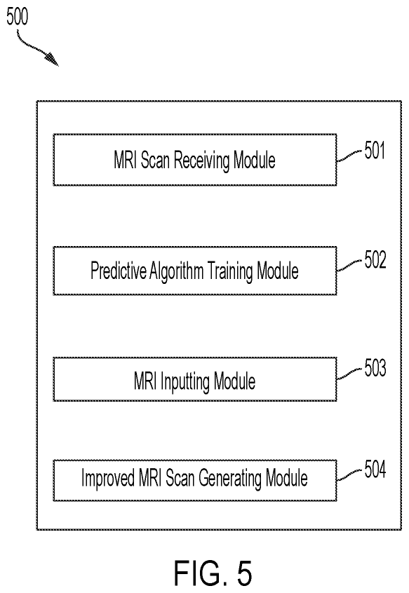

Turning ahead in the drawings, FIG. 4 illustrates a flow chart for a method 400, according to an embodiment. Method 400 is merely exemplary and is not limited to the embodiments presented herein. Method 400 can be employed in many different embodiments or examples not specifically depicted or described herein. In some embodiments, the activities of method 400 can be performed in the order presented. In other embodiments, the activities of method 400 can be performed in any suitable order. In still other embodiments, one or more of the activities of method 400 can be combined or skipped. In some embodiments, one or more elements of system 100 (FIG. 1), system 300 (FIG. 3), and/or system 500 (FIG. 5) can be suitable to perform method 400 and/or one or more of the activities of method 400. In these or other embodiments, one or more of the activities of method 400 can be implemented as one or more computer instructions configured to run at one or more processing modules and configured to be stored at one or more non-transitory memory storage modules. Such non-transitory memory storage modules can be part of a computer system such as web server 301 (FIG. 3), MRI scanner 302 (FIG. 3), and/or electronic device 303 (FIG. 3). The processing module(s) can be similar or identical to the processing module(s) described above with respect to computer system 100 (FIG. 1).

In some embodiments, method 400 can comprise an activity 401 of receiving an MRI scan. In some embodiments, activity 401 can comprise a vessel wall imaging protocol including a pre-contrast scan and a post-contrast scan. In various embodiments, an MRI can be received from one or more of web server 301 (FIG. 3) and/or MRI scanner 302 (FIG. 3). For example, an MRI scan can comprise a scan taken of a patient. As another example, an MRI scan can comprise one or more training MRI scans and/or a training data set used to train a predictive algorithm. In some embodiments, MRI scans received in activity 401 can comprise raw MRI data. In some embodiments, raw MRI data can comprise a raw data matrix. Generally speaking, a raw data matrix can comprise data collected by the MRI machine's receiver coils during an MRI scanning process. In various embodiments, a raw data matrix can comprise a matrix of numbers that represents an amplitude and phase of signals received from a patient at an MRI scanner's (e.g., MRI scanner 302's (FIG. 3)) radio frequency coils. In further embodiments, a raw data matrix can contain a sufficient amount of information needed to reconstruct an image of the patient but cannot be directly interpreted as an image due to its raw formatting. In various embodiments, a raw data matrix can comprise an amplitude, a phase, a frequency, and/or a time entry. In some embodiments, an amplitude can comprise an amplitude of a signal received by an RF coil. In some embodiments, a phase can comprise a phase of hydrogen atoms in the body (e.g., a frequency of spin and/or precess for the hydrogen atoms). In some embodiments, time can comprise a time at which a signal was received by an RF coil. In some embodiments, raw MRI data can comprise data in a k-space. Generally speaking, k-space can comprise a mathematical space generated by MRI scans that contains a processed version of a raw data matrix. In some embodiments, k-space data can be understood as a 2D or 3D grid representing a frequency and phase of radio signals received by an RF coil. A raw data matrix can be converted into a k-space in a number of ways. For example, a Fourier transform can be applied to a raw data matrix, data in a raw data matrix can be filtered (e.g., by frequency), a correction can be applied to a phase of data in a raw data matrix, magnetic gradients present in a raw data matrix can be encoded to map a signal to a specific location in a patient, and/or a reverse Fourier transform can be applied to transform data into an image domain. In various embodiments, these exemplary steps can be performed in sequence to generate an image of a vessel. In various embodiments, a raw data matrix and/or k-space data can be modified to produce intensity values for each pixel in an MRI scan or some other value to convey pixel information (e.g., RGB content).

In some embodiments, method 400 can optionally comprise an activity 402 of training a predictive algorithm. In various embodiments (e.g., when a pre-trained algorithm is used), activity 402 can be skipped. In some embodiments, training a predictive algorithm can comprise estimating internal parameters of a model configured to segment an MRI scan. In various embodiments, a predictive algorithm can be trained using training data, otherwise known as a training dataset. In some embodiments, a training dataset can comprise all or a part of an MRI scan received in activity 401. In this way, a predictive algorithm can be configured to classify a pixel in an MRI scan and/or segment an MRI scan into different segments. In the same or different embodiments, a predictive algorithm can comprise a neural network, as described in further detail below.

A pre-trained predictive algorithm can be used, and the pre-trained algorithm can be re-trained on the training data. In some embodiments, a predictive algorithm can also consider both historical and newly added MRI scans when making a prediction. In this way, a predictive algorithm can be trained iteratively as data from MRI scans are added to a training data set. In various embodiments, a predictive algorithm can be trained, at least in part, on a single patient's MRI scans or the single patient's MRI scans can be weighted in a training data set. In this way, a predictive algorithm tailored to a single patient can be generated. A predictive algorithm tailored to a single patient can be used as a pre-trained algorithm for a similar patient. In various embodiments, due to a large amount of data needed to create and maintain a training data set, a predictive algorithm can use extensive data inputs to enhance and/or improve an MRI scan. Due to these extensive data inputs, in some embodiments, creating, training, and/or using a predictive algorithm configured to enhance and/or improve an MRI scan cannot practically be performed in a mind of a human being. A predictive algorithm can estimate inner and outer vessel wall boundaries simultaneously.

Training data and/or a training dataset can take a number of forms. For example, training data can be labeled and/or unlabeled. In some embodiments, labeled training data can comprise a dataset in which each data point has an associated label or output value that represents a correct prediction for that data point. For example, in a dataset of MRI scans, pixels in the MRI scans can be labeled as lumen, vessel wall, or background. Unlabeled training data can comprise a dataset in which pixels do not have associated label or output value. For example, a dataset of MRI scans without labels would be considered unlabeled. Generally speaking, labeled data provides explicit guidance to a predictive algorithm during training while unlabeled data does not provide explicit guidance. In some embodiments, all or a portion of a training dataset can be labeled as a ground truth (e.g., as ground truth lumen, ground truth vessel wall, or ground truth background). Generally speaking, ground truth labels can be understood as a correct or known label assigned to training data. In various embodiments, ground truth labels can be used during activity 402 to teach a predictive algorithm to identify patterns and relationships between input data and corresponding output predictions. In various embodiments, an accuracy of a trained predictive algorithm can be evaluated on a testing dataset with known ground truth labels that are hidden from the predictive algorithm.

In some embodiments, training dataset can comprise T1-weighted MR VWIs from 80 patients diagnosed with ICAD. In some embodiments, an intracranial internal carotid artery, a middle cerebral artery, an intracranial vertebral artery, or a basilar artery can be included as segmented samples in training data. In various embodiments, lumens provided as training data can have plaque involvement. In various embodiments, contiguous 2D cross-sectional slices with 0.55 mm slice thickness and 0.1 mm in-plane resolution from each segment can be generated, and a ground truth lumen and vessel wall can be labeled by a radiologist.

An objective function can be used in activity 402 to train a predictive algorithm. Generally speaking, an objective function (also known as a loss function or cost function) can be used to measure how well a predictive algorithm is performing on a given task. In various embodiments, an objective function can take in a predicted output of a predictive algorithm (e.g., a an improve MRI scan) and compare it to a true output (e.g., a ground truth label). In various embodiments, a predictive algorithm can adjust its parameters to minimize a value of an objective function. In further embodiments, a backpropagation technique can be used in combination with an objective function to train a predictive algorithm. In some embodiments, a backpropagation technique can involve calculating a gradient of an objective function with respect to parameters of a predictive algorithm. Once a gradient is calculated, it can be used to update the parameters in an opposite direction of the gradient and/or with a gradient. By iteratively adjusting the parameters in this way, a predictive algorithm can learn to make more accurate predictions.

An objective function can take a number of forms. For example, an objective function can comprise a cross-entropy loss, which measures the difference between predicted class probabilities and true class probabilities or a mean squared error, which measures the difference between predicted values and true values. In some embodiments, an objective function can comprise three terms: a fidelity term configured to match derived level-sets with training labels and two regularization terms configured to encourage smoothness for a predicted value function and class boundaries, respectively. In this way, smoothness of segmentation boundaries predicted by a predictive algorithm can be regularized. In various embodiments, a loss function can be understood as a summation of these three terms that is weighted by hyperparameters λ and γ. In some embodiments, a loss function can be formulated as:

$$L = {L}^{fidelity} + λ {L}^{s m o o t h} + γ {L}^{L e n g t h}$$

In some embodiments, L can comprise a loss, L^{fidelity} can comprise a fidelity term, L^{smooth} can comprise a smoothness term, and L^{Length} can comprise a class boundaries term. In some embodiments, a fidelity term can define an agreement between a predicted label and a given labels using a soft Dice criterion for lumen, vessel wall, and background classes. In various embodiments, an I₂ norm can be applied to a gradient of network output y. In this way, clear and robust differentiation between adjacent classes (e.g., lumen vs. vessel wall and vessel wall vs. background) can be created, oscillations can be reduced, and region-wise stability and homogenous membership can be promoted. In these embodiments, L^{Length} can be formulated as:

$${L}^{L e n g t h} = \frac{1}{N} \sum {‖ ∇ y ‖}^{2} ,$$

where N can comprise a total number of pixels analyzed.

In some embodiments, when a contour of a lumen is approached, a length penalty based on total variation can be imposed on a vessel wall class to reduce a roughness of inner and outer boundaries of the vessel wall. In these embodiments, L^{Length} can be formulated as:

$${L}^{L e n g t h} = \frac{1}{N} \sum ‖ ∇ {y}_{vessel \_ wall}^{′} ‖ ,$$

where y′_{vessel_wall} can comprise a continuous probability-like relaxation of a network output y for pixels labeled as a vessel wall class.

In some embodiments, method 400 can comprise an activity 403 of inputting an MRI scan into a predictive algorithm. In some embodiments, an MRI scan can comprise an MRI scan received in activity 401 and/or an MRI scan collected from a patient. In various embodiments, one or more portions of an MRI scan can be concatenated to form a vector, which can then be inputted into a predictive algorithm. A predictive algorithm can comprise a segmentation algorithm configured to determine which portions of an MRI are vessel wall, lumen, and/or background. Generally speaking, a segmentation algorithm can be configured to partition an image into multiple regions or segments. In some embodiments, segments can be labeled with one or more of vessel wall, lumen, and/or background.

A predictive algorithm can comprise a neural network. Generally speaking, aneural network is a type of machine learning algorithm modeled after the structure and function of the human brain. In various embodiments, a neural network can comprise layers of interconnected neurons. In some embodiments, data input into a neural network can be fed into a first layer and then passed through multiple layers (e.g., hidden layers) of neurons. In some embodiments, an output can be generated at a final layer. Each neuron in a neural network can apply a mathematical operation to data it receives before passing an output for that neuron to a subsequent neuron. In some embodiments, connections between neurons (known as synapses) have a weight that can be adjusted during training. In this way, a performance of the neural network can be optimized. In some embodiments, training a neural network can comprise adjusting weights of synapses so that the network can accurately predict a shape of a bodily structure (e.g., a lumen of a vessel).

In some embodiments, a convolutional neural network (CNN) can be used. Generally speaking, a CNN is a type of neural network designed to learn spatial hierarchies of features automatically and adaptively from input data. In some embodiments, a CNN can comprise a 2.5D UNet model with ResNet backbone. In some embodiments, a CNN can have a single-channel-output. In this way, an inclusion relationship can be incorporated into an optimization problem solved by the CNN. A single-channel-output can further enable a soft tiered relationship between a lumen, vessel, and background segment. A CNN can comprise a convolutional layer, a pooling layer, a deconvolutional layer, and/or a fully connected layer. Generally speaking, convolutional layers perform feature extraction using a convolution operation, pooling layers downsample feature maps to reduce a size of data in the network (and thereby increase computational efficiency), deconvolutional layers upsample feature maps to improve further improve the network's ability to learn complex features, and fully connected layers can be used to produce an output of the network.

In some embodiments, a convolutional layer can implement a type of mathematical operation called a convolution that extracts relevant features from input data. In some embodiments, a convolutional layer can perform convolutions on an MRI scan. In some embodiments, a convolution involves sliding a small matrix (referred to as a kernel or a filter) over input data and performing a dot product between the kernel and the input data. In various embodiments, an output of a convolution can comprise a set of feature maps, each representing a specific feature in the input data. In some embodiments, a feature map can be passed through an activation function (e.g., a ReLU). An output of an activation function can be passed to a next node or layer in a CNN. In some embodiments, a convolution portions of a CNN can comprise one convolution layer followed by a batch normalization layer, and then another convolution layer. A CNN can have a single channel output via a lxl convolution layer with a sigmoid activation function. In this way, a single-channel prediction can map each pixel's value to its corresponding class membership.

In some embodiments, a CNN can implement a level-set algorithm. Generally speaking, a level-set algorithm can be seen as a statistical technique that uses a level-set function to define a boundary between an object of interest and a background. In some embodiments, a level-set algorithm can be configured to evolve a boundary of a lumen of interest until it converges to an actual boundary of the lumen. When implemented on a neural network, a level-set function can cause the neural network to predict whether a pixel in an MRI scan is a member of a lumen, vessel wall, and/or a background. A level-set algorithm can begin by initially estimating a vessel boundary (e.g., by specifying a set of seed points or by using an existing segmentation algorithm). In various embodiments, after a vessel boundary has been estimated, a level-set function can be defined to represent the boundary. A level-set function can then be evolved over time using a partial differential equation (PDE). In various embodiments, a PDE can be configured to cause a level-set function to move inward or outward from a vessel boundary depending on an image gradient at pixel. In this way, a level-set function can converge to an actual object boundary.

In some embodiments, inclusion relationships between a vessel and lumen can be incorporated via level-set function heights. In some embodiments, an inclusion can be encoded into a level-set algorithm by considering ordinal relations among various level-set functions with respect to a single level-set function. Under a 2D setting, a level-set function can be defined as:
Φ(x): R²→R, where Φ(x) can comprise a level-set function mapping a pixel x from two-dimensional vector space R² to a one-dimensional vector space R.

In various embodiments lumen and vessel pixels can be defined as:
Ωₗᵤₘₑₙ{x: Φ(x), η₁},
Ω_{whole_vessel}{x: Φ(x), η₂}, where Ωₗᵤₘₑₙ can comprise pixels labeled as a lumen, Ω_{whole_vessel} can comprise pixels labeled as a vessel, η₁ can comprise a first pre-defined parameter, and η₂ can comprise a second pre-defined parameter. In some embodiments, η₁=⅓ and η₂=⅔.

This formulation of equations leverages the relation that, for η₁<η₂, Ωₗᵤₘₑₙ⊂η_{whole_vessel} reflects an inclusion relationship. In some embodiments, a background can be defined as a complement of a larger set of pixels:

Ω_{background}=D−Ω_{whole_vessel}, where D∈R² denotes an entire segmentation domain and Ω_{background} can comprise pixels labeled as background.

In some embodiments, a membership of a pixel as a vessel, lumen, or background can be obtained by taking a level-set function through a Heaviside function H:

$$\{ \begin{matrix}H ( {η}_{1} - Φ ( x ) ) = 1 , x ∈ {Ω}_{lumen} \\ H ( {η}_{2} - Φ ( x ) ) \cdot H ( Φ ( x ) - {η}_{1} ) = 1 , x ∈ {Ω}_{vessel \_ wall} \\ H ( Φ ( x ) - {η}_{2} ) = 1 , x ∈ {Ω}_{b a c k g r ound}\end{matrix}$$

In some embodiments, a level-set function taken through a Heaviside function can be relaxed to a continuous differential sigmoid function using the equation below:

$$S ( x ) = \frac{1}{1 + {e}^{- x}}$$

Corresponding continuous probability-like relaxation of the network predicted value function y to y′ is given by:

$$\{ \begin{matrix}{y}_{lumen}^{′} = S ( {η}_{1} - y ) , \\ {y}_{vessel \_ wall}^{′} = S ( {η}_{2} - y ) \cdot S ( y - {η}_{1} ) \\ S ( y - {η}_{2} )\end{matrix} ,$$

In some embodiments, a CNN can implement a skip connection. Generally speaking, a skip connection can be described as a connection between two or more layers in a neural network that allows the network to skip a layer in a flow of the network. In some embodiments, a skip connection can be inserted after each convolutional layer. This skip layer can allow a signal to be passed through a 1×1 convolution to add a feature of a previous layer to a last layer of a convolutional block.

In some embodiments, method 400 can comprise an activity 404 of generating an improved MRI scan. In various embodiments, an improved MRI scan can comprise a more real to life reconstruction of an MRI scan as described in activity 401. For example, an improved MRI scan can have sharper delineations between different layers, organs, and/or structures of a patient's body. As a further example, an improved MRI scan can have smoother lines and/or edges. As another example, an improved MRI scan can have fewer or zero artifacts. In some embodiments, an improved MRI scan can be displayed on a GUI (e.g., a GUI as described in FIG. 3).

Turning ahead in the drawings, FIG. 5 illustrates a block diagram of a system 500 that can be employed for behavior based messaging. System 500 is merely exemplary and embodiments of the system are not limited to the embodiments presented herein. System 500 can be employed in many different embodiments or examples not specifically depicted or described herein. In some embodiments, certain elements or modules of system 500 can perform various procedures, processes, and/or activities. In these or other embodiments, the procedures, processes, and/or activities can be performed by other suitable elements or modules of system 500. In some embodiments, one or more portions of system 500 can be part of or in communication with web server 301 (FIG. 3), MRI scanner 302 (FIG. 3), and/or electronic device 303 (FIG. 3).

Generally, therefore, system 500 can be implemented with hardware and/or software, as described herein. In some embodiments, part or all of the hardware and/or software can be conventional, while in these or other embodiments, part or all of the hardware and/or software can be customized (e.g., optimized) for implementing part or all of the functionality of system 500 described herein.

In some embodiments, system 500 can comprise non-transitory memory storage module 501. Memory storage module 501 can be referred to as MRI scan receiving module 501. In some embodiments, MRI scan receiving module 501 can store computing instructions configured to run on one or more processing modules and perform one or more acts of method 400 (FIG. 4) (e.g., activity 401 (FIG. 4)).

In some embodiments, system 500 can comprise non-transitory memory storage module 502. Memory storage module 502 can be referred to as predictive algorithm training module 502. In some embodiments, predictive algorithm training module 502 can store computing instructions configured to run on one or more processing modules and perform one or more acts of method 400 (FIG. 4) (e.g., activity 402 (FIG. 4)).

In some embodiments, system 500 can comprise non-transitory memory storage module 503. Memory storage module 503 can be referred to as MRI inputting module 503. In some embodiments, MRI inputting module 503 can store computing instructions configured to run on one or more processing modules and perform one or more acts of method 400 (FIG. 4) (e.g., activity 403 (FIG. 4)).

In some embodiments, system 500 can comprise non-transitory memory storage module 504. Memory storage module 504 can be referred to as improved MRI scan generating module 504. In some embodiments, improved MRI scan generating module 504 can store computing instructions configured to run on one or more processing modules and perform one or more acts of method 400 (FIG. 4) (e.g., activity 404 (FIG. 4)).

Although systems and methods for automated vessel wall segmentation have been described with reference to specific embodiments, it will be understood by those skilled in the art that various changes may be made without departing from the spirit or scope of the disclosure. Accordingly, the disclosure of embodiments is intended to be illustrative of the scope of the disclosure and is not intended to be limiting. It is intended that the scope of the disclosure shall be limited only to the extent required by the appended claims. For example, to one of ordinary skill in the art, it will be readily apparent that any element of FIGS. 1-5 may be modified, and that the foregoing discussion of certain of these embodiments does not necessarily represent a complete description of all possible embodiments. For example, one or more of the procedures, processes, or activities of FIG. 4 may include different procedures, processes, and/or activities and be performed by many different modules, in many different orders.

All elements claimed in any particular claim are essential to the embodiment claimed in that particular claim. Consequently, replacement of one or more claimed elements constitutes reconstruction and not repair. Additionally, benefits, other advantages, and solutions to problems have been described with regard to specific embodiments. The benefits, advantages, solutions to problems, and any element or elements that may cause any benefit, advantage, or solution to occur or become more pronounced, however, are not to be construed as critical, required, or essential features or elements of any or all of the claims, unless such benefits, advantages, solutions, or elements are stated in such claim.

Moreover, embodiments and limitations disclosed herein are not dedicated to the public under the doctrine of dedication if the embodiments and/or limitations: (1) are not expressly claimed in the claims; and (2) are or are potentially equivalents of express elements and/or limitations in the claims under the doctrine of equivalents.

What is claimed is:

1. An automated vessel wall segmentation system to classify pixels in an image of a vessel wall, the system comprising:
a processor; and
a non-transitory computer-readable storage devices storing computing instructions configured to run on the processor and cause the processor to perform:
receiving a magnetic resonance imaging (MRI) scan;
feeding the MRI scan into a predictive algorithm; and
outputting an improved MRI scan from the predictive algorithm.

2. The system of claim 1, wherein the predictive algorithm comprises:
a first convolution layer having a first input and first output and receiving data representative of the image at the first input;
a batch normalization layer following the first convolution layer and connected to the first output of the first convolution layer;
a second convolution layer following the batch normalization layer and having a second input and second output, the second input connected to the batch normalization layer; and
a skip connection connected between the first input and the second input, the skip connection including a neural network;
wherein the second output is a single channel output providing data representing a predicted classification of a pixel as corresponding to one of (i) a lumen, (ii) a background, and (iii) the vessel wall.

3. The system of claim 2, wherein the neural network uses an objective function comprising three terms:
(i) a fidelity term to match a derived level-sets with training labels,
(ii) a first regularization term to promote smoothness for a value function associated with the pixel, and
(iii) a second regularization term to promote smoothness for a class boundary defined between at least two of (i) the lumen, (ii) the background, and (iii) the vessel wall.

4. The system of claim 3, wherein the fidelity term defines an agreement between the predicted classification and an actual classification of the pixel using a soft Dice criterion.

5. The system of claim 1, wherein the improved MRI scan has smoother segmentation boundaries between a vessel, a lumen, and a background than on the MRI scan.

6. The system of claim 1, wherein the predictive algorithm comprises a convolutional neural network having a lxi convolution layer.

7. The system of claim 1, wherein the computing instructions are further configured to run on the processor and cause the processor to perform:
training the predictive algorithm on T1 weighted MRI scans.

8. The system of claim 1, wherein the predictive algorithm incorporates a length penalty on a vessel wall class of pixel.

9. A method for automated vessel wall segmentation that classifies pixels in an image of a vessel wall, the method comprising:
receiving a magnetic resonance imaging (MRI) scan;
feeding the MRI scan into a predictive algorithm; and
outputting an improved MRI scan from the predictive algorithm.

10. The method of claim 9, wherein the predictive algorithm comprises:
a first convolution layer having a first input and first output and receiving data representative of the image at the first input;
a batch normalization layer following the first convolution layer and connected to the first output of the first convolution layer;
a second convolution layer following the batch normalization layer and having a second input and second output, the second input connected to the batch normalization layer; and
a skip connection connected between the first input and the second input, the skip connection including a neural network;
wherein the second output is a single channel output providing data representing a predicted classification of a pixel as corresponding to one of (i) a lumen, (ii) a background, and/or (iii) the vessel wall.

11. The method of claim 10, wherein the neural network uses an objective function comprising three terms:
(i) a fidelity term to match a derived level-sets with training labels,
(ii) a first regularization term to promote smoothness for a value function associated with the pixel, and
(iii) a second regularization term to promote smoothness for a class boundary defined between at least two of (i) the lumen, (ii) the background, and (iii) the vessel wall.

12. The method of claim 11, wherein the fidelity term defines an agreement between the predicted classification and an actual classification of the pixel using a soft Dice criterion.

13. The method of claim 9, wherein the improved MRI scan has smoother segmentation boundaries between a vessel, a lumen, and a background than on the MRI scan.

14. The method of claim 9, wherein the predictive algorithm comprises a convolutional neural network having a lxi convolution layer.

15. The method of claim 9 further comprising training a predictive algorithm on T1 weighted MRI scans.

16. The method of claim 9, wherein the predictive algorithm incorporates a length penalty on a vessel wall class of pixel.

17. A method of automated vessel wall segmentation to classify a pixel in an image of a vessel wall, the method comprising:
receiving data representative of the image of at least one of (i) a lumen, (ii) a background, and/or (iii) the vessel wall at a first input of a first convolution layer, the first convolution layer having a first output connected to a batch normalization layer and providing a first output data at the first output;
normalizing, by the batch normalization layer, the first output data to create normalized output data;
providing, by the batch normalization layer, the normalized output data to a second convolution layer having a second input to receive the normalized output data and having a second output;
maximizing, by a skip connection including a neural network connected between the first input and the second input, a function comprising at least a fidelity term, a first regularization term, and a second regularization term and providing the maximized function to the second convolution layer;
calculating, by the second convolution layer, a second output data based on the normalized output data and the function, the second output data representing a predicted classification of the pixel as corresponding to at least one of (i) the lumen, (ii) the background, and/or (iii) the vessel wall; and
providing, by the second convolution layer, the second output data at the second output.

18. The method of claim 17, wherein the second output is a single channel output.

19. The method of claim 17, wherein the fidelity term matches derived level-sets with training labels.

20. The method of claim 17, wherein the first regularization term promotes smoothness for a value function associated with the pixel.
